(19) United States
(12) Patent Application Publication
(10) Pub. No.: US 2024/0306857 A1
(43) Pub. Date: Sep. 19, 2024

(54) SOLID-STATE FILM DISPENSER AND DELIVERY DEVICE THEREFORE

(71) Applicant: Joshua Stancle, Sherman Oaks, LA (US)

(72) Inventor: Joshua Stancle, Sherman Oaks, LA (US)

(21) Appl. No.: 18/673,643

(22) Filed: May 24, 2024

(51) Int. Cl. A47K 5/12; A61J 7/00; A61M 35/00

(52) U.S. Cl. CPC A47K 5/1201; A61J 7/0053; A61M 35/003

(57) ABSTRACT

The invention discloses a dispenser device tailored for administering solid-state film compositions. This device consists of a housing equipped with an outlet and a mechanism adept at propelling the solid-state composition through the outlet. The mechanism boasts adjustability to precisely dispense dosages of the solid-state film composition. Crucially, the device integrates a user interface enabling users to select their desired dosage of the solid-state film composition for dispensing. This innovation offers a convenient and customizable solution for dispensing solid-state film compositions, spanning toothpaste formulations, dietary supplements, skincare formulations, and pharmaceutical formulations, enhancing user experience and dosage accuracy.

### FIELD OF THE INVENTION

The present invention relates generally to compositions and devices for oral health and overall wellness. More specifically, the present invention relates to solid-state film toothpaste, dietary supplements, skincare, and pharmaceuticals compositions and devices to dispense or deliver the solid-state film formulations of toothpaste, supplements, skincare, and pharmaceuticals.

### BACKGROUND OF THE INVENTION

Maintaining or enhancing oral health and wellness holds paramount significance in overall human well-being. A prime example underscores the repercussions of inadequate oral care, where neglect can precipitate gum disease, notably gingivitis, eventually culminating in tooth loss. Such ramifications extend beyond mere dental concerns, profoundly impacting an individual's holistic health and quality of life. Compromised oral health not only diminishes physical comfort and functionality but also exacerbates systemic health issues. The interconnection between oral health and general well-being underscores the urgent need for effective preventive measures and therapeutic interventions. By addressing oral health comprehensively, including through innovative solutions such as the dispenser device for solid-state film compositions, we can mitigate risks, improve outcomes, and foster enduring wellness for individuals across diverse demographics.

The primary culprits contributing to tooth loss encompass tooth decay and gum disease, posing substantial risks that amplify with age. Several factors exacerbate these risks over time. Firstly, issues with saliva production can compromise oral health, as saliva plays a crucial role in rinsing away food particles and neutralizing acids that contribute to decay. Additionally, age-related phenomena like receding gums expose vulnerable root surfaces to bacteria, heightening susceptibility to decay. Challenges in maintaining oral hygiene further compound these risks, often stemming from age-related factors such as deteriorating vision, cognitive decline, chronic illnesses, and physical limitations. Consequently, difficulties in flossing and brushing effectively arise, facilitating the accumulation of plaque and bacterial growth. Recognizing these multifaceted challenges underscores the imperative for tailored oral care strategies and innovative solutions to mitigate the impact of age-related factors on oral health outcomes.

In the United States, despite improvements in oral hygiene practices among adults, a significant portion still requires dental treatment, reflecting pervasive dental issues. Shockingly, over a quarter of adults (26%) grapple with untreated tooth decay, underscoring the pressing need for intervention to avert broader health consequences stemming from decay. Furthermore, the prevalence of gum disease is alarmingly high, with nearly half (46%) of adults aged 30 or older exhibiting signs of this condition, while severe gum disease afflicts approximately 9% of adults. One contributing factor to these concerning statistics is the logistical challenges individuals face in maintaining oral hygiene routines while on the go or after meals. The inconvenience of carrying traditional toothpaste and a toothbrush, coupled with the requirement for access to a sink and running water, presents formidable barriers that hinder timely oral care practices.

These obstacles not only impede immediate oral hygiene efforts but also exacerbate the risk of dental problems and undermine overall health and wellness. The inability to address oral hygiene needs promptly and effectively leaves individuals vulnerable to the progressive deterioration of oral health, potentially culminating in severe dental issues and compromised overall well-being. Consequently, there is a critical need for innovative solutions that transcend traditional oral care approaches, offering convenience and efficacy to facilitate consistent and accessible oral hygiene practices, particularly in scenarios where conventional methods prove impractical or inaccessible.

Concurrently, when users do brush their teeth, a vast majority of users use far too much toothpaste than necessary in order to clean one's teeth. This can have negative consequences apart from the clear burden of the increasing cost of toothpaste, such as for example, fluoride, when ingested in large amounts, can cause a cosmetic condition known as dental fluorosis on one's developing teeth. The cosmetic implications range from mild discoloration to yellow and brown stains to obvious pits in the teeth

Also, the recommended amount of toothpaste differs for users, such as between adults and children. It is difficult for users to ascertain the recommended amount of toothpaste to use based on their particular needs.

Consequently, what is needed is a composition and device for obviating the above-described problems in addition to addressing skin care needs, dietary supplementation, and pharmaceutical dispensing.

### SUMMARY OF THE INVENTION

To address the foregoing problems, in whole or in part, and/or other problems that may have been observed by persons skilled in the art, the present disclosure provides compositions and methods as described by way of example as set forth below.

A principal objective of the invention is to develop a dispenser device that is compact, lightweight, and easily portable, enabling users to maintain oral hygiene routines while on the go, without the need for access to a sink or running water.

Another objective of the invention is to implement a mechanism within the dispenser device that allows for accurate and adjustable dosing of the solid-state film composition, ensuring users can dispense the appropriate amount tailored to their specific needs and preferences.

Another objective of the invention is to utilize materials in the construction of the dispenser device that are non-reactive with the solid-state composition, ensuring compatibility and safety during storage and dispensing, thereby safeguarding the integrity and efficacy of the compositions.

Another objective of the invention is to incorporate a user-friendly interface into the dispenser device, enabling intuitive selection of the desired dosage and type of solid-state film composition to be dispensed, enhancing usability and accessibility for users of diverse demographics and preferences.

In view of the foregoing, the present invention provides a dispenser device for administering a solid-state film composition. The device comprises a housing having an outlet, and a mechanism configured to advance the solid-state composition through an outlet of the housing. The mechanism is adjustable to dispense precise dosages of the solid-state film composition and the device comprises a user interface allowing users to select the dosage of solid-state film composition to be dispensed.

In an aspect of the invention, the solid-state film composition comprises any one of a toothpaste formulation, or one or more dietary supplements configured for oral ingestion, or a skincare formulation for topical application, or a pharmaceutical formulation for medication delivery.

In an aspect of the invention, the housing and mechanism are composed of materials non-reactive with the solid-state film composition.

In an aspect of the invention, the housing comprises a replaceable cartridge or film roll containing the solid-state film composition.

In an aspect of the invention, the mechanism comprises a splitter or a cutter to split or cut the solid-state film composition in response to user input.

In an aspect of the invention, the mechanism comprises a rotary gear system for precise control of the solid-state composition dispensing.

In an aspect of the invention, a sensor system to detect the presence of the solid-state composition within the housing and provide feedback to the user interface for dosage adjustment.

In an aspect of the invention, the housing is made of a UV-resistant material to protect the solid-state composition from degradation due to light exposure.

In an aspect of the invention, the device may comprise a solid-state film toothpaste, dietary supplements, skincare, and pharmaceuticals cartridge, or film roll that is replaceable in order to ensure easy replacement and continuous use.

In an aspect, the dispenser device may be a single use dispenser.

In an aspect, a sealed compartment protects the solid-state film toothpaste, dietary supplements, skincare, and pharmaceuticals from environmental contaminants and germs, ensuring hygiene during use.

In an aspect, the device provides portability and travel access without limitations to standard travel ounce restrictions.

In an aspect, a splitter or cutter is provided to split or cut the solid-state film toothpaste, dietary supplements, skincare, and pharmaceuticals in response to an input from a user to ensure proper size and amount.

In an aspect, the compositions for use in the device may be produced with glycerol, gelatin, flavoring, vitamins/minerals, glucose, purified water, sodium citrate, pharmaceutical, citric acid, breath freshening components, and various other dental hygiene ingredients described herein.

In an aspect, the composition in its solid-state film or gel form may comprise a combination of pullulan, menthol, sucralose, glycerol oleate, copper gluconate, ceratonia siliqua, though other ingredients are contemplated.

In an aspect, the composition comprises compounds and ingredients that have direct therapeutic benefits (e.g., active ingredients) and other components or ingredients that act as carriers or taste modifying ingredients (e.g., inactive ingredients).

In an aspect, the composition comprises the active ingredients in the range from about 10.0% to about 40.0% by weight and inactive ingredients in the amount of 60% to about 90% by weight. In one embodiment, the composition comprises fluoropolymers such as for example, polytetrafluoroethylene (PTFE), fluorinated polyethylene-propylene (FEP), polyvinylidene fluoride (PVDF), and polyvinylfluoride (PVF). In embodiments, other anti-caries agents, which enhance the dental remineralization process and help repair early tooth decay, may be used.

In an aspect, antimicrobial agents such as stannous fluoride, triclosan, and zinc citrate, which help to prevent the accumulation of plaque, may be employed.

In an aspect, flavorings such as peppermint, spearmint, cinnamon, wintergreen, and menthol may be used to freshen breath and add taste may be used.

In an aspect, culturally specific flavorings may be used and sweeteners such as xylitol, calcium or sodium saccharin and aspartame may be employed.

In an aspect, the composition may be a microcrystalline cellulose and maltodextrin composition as a solid-state film, which can be quickly and completely digestible by the user.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 shows a schematic view of a dispenser device for administering a solid-state film composition in accordance with embodiments of the present disclosure;

FIG. 2 is a front view of a device to dispense a solid-state film toothpaste, dietary supplements, skincare, and pharmaceuticals and the solid-state toothpaste in the device in accordance with embodiments of the present disclosure;

FIG. 3 is a vertical sectional view of a device to dispense a solid-state film toothpaste, dietary supplements, skincare, and pharmaceuticals and the solid-state toothpaste in the device in accordance with embodiments of the present disclosure;

FIG. 4 is another vertical cross section view of a device to dispense a solid-state film toothpaste, dietary supplements, skincare, and pharmaceuticals and the solid-state toothpaste in the device in accordance with embodiments of the present disclosure;

FIG. 5 is a side cross section view of a device to dispense a solid-state film toothpaste, dietary supplements, skincare, and pharmaceuticals and the solid-state toothpaste in the device in accordance with embodiments of the present disclosure.

FIG. 6 and FIG. 7 is a device to dispense a solid-state film toothpaste, dietary supplements, skincare, and pharmaceuticals and the solid-state toothpaste with detailed components.

FIG. 8A and FIG. 8B is a cross-section view of the dispenser device, in accordance with embodiments of the present disclosure.

Skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having the benefit of the description herein.

### DETAILED DESCRIPTION OF THE INVENTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and example of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one”, but does not exclude a plurality unless the contextual use dictates otherwise. When used herein to join a list of items, “or” denotes “at least one of the items”, but does not exclude a plurality of items of the list. Finally, when used herein to join a list of items, “and” denotes “all of the items of the list”.

The present invention discloses a dispenser device designed to administer solid-state film compositions efficiently and conveniently. At its core, the device features a housing equipped with an outlet, serving as the conduit for dispensing the solid-state composition. A pivotal aspect of the device lies in its mechanism, ingeniously engineered to propel the solid-state composition through the outlet with precision and control. This mechanism is adjustable, empowering users to dispense precise dosages of the solid-state film composition tailored to their specific needs and preferences. Crucially, the device incorporates a user interface, enhancing user interaction by providing intuitive controls for selecting the desired dosage of the solid-state film composition to be dispensed.

In essence, the dispenser device revolutionizes the administration of solid-state film compositions, offering unparalleled convenience and versatility. Its compact housing and portable design enable users to maintain their oral hygiene routines effortlessly, whether at home or on the go, eliminating the need for cumbersome traditional oral care products. The adjustable mechanism ensures dosage accuracy, empowering users to dispense the exact amount of the solid-state composition required for optimal oral health. Moreover, the inclusion of a user interface enhances usability, allowing for seamless interaction and customization of dosage preferences, thus catering to the diverse needs of users across demographics. Overall, this innovative device sets a new standard in oral care, simplifying and enhancing the user experience while promoting oral health and wellness.

In an embodiment, the solid-state film composition featured in the invention encompasses a versatile range of formulations tailored to address various aspects of personal care and wellness. Firstly, serving as a toothpaste formulation, it provides an innovative alternative to conventional toothpaste for maintaining oral hygiene. By encapsulating essential dental cleaning agents, the solid-state film ensures effective plaque removal and protection against tooth decay and gum disease, promoting overall oral health with convenience and efficacy.

Moreover, extending its utility beyond oral care, the composition incorporates dietary supplements configured for oral ingestion. This inclusion offers users a convenient method of supplementing essential nutrients and vitamins, supporting overall health and vitality. Additionally, the composition includes skincare formulations designed for topical application, delivering targeted solutions for skin health and rejuvenation. By providing hydration, nourishment, and anti-aging benefits in a solid-state film format, the composition offers users a convenient and mess-free approach to skincare. Furthermore, the incorporation of pharmaceutical formulations for medication delivery enhances the versatility of the invention, facilitating precise and convenient administration of medications to address specific medical needs or conditions.

In an embodiment, the housing and mechanism of the dispenser device are meticulously crafted from materials specifically chosen for their non-reactive properties with the solid-state film composition. This design consideration is paramount to ensuring the integrity and efficacy of the compositions dispensed by the device. By utilizing materials that do not react with the solid-state film, the risk of contamination, degradation, or alteration of the composition's properties is effectively mitigated, safeguarding its therapeutic or hygienic benefits.

Furthermore, the choice of non-reactive materials for the housing and mechanism enhances the overall safety and reliability of the device. Users can trust that the materials used in its construction will not introduce any harmful substances or compromise the quality of the solid-state film composition. This aspect is particularly crucial in applications involving oral hygiene, skincare, or medication delivery, where product purity and safety are paramount concerns.

In an embodiment, housing of the invention incorporates a pivotal element crucial for its functionality-a replaceable cartridge or film roll that contains the solid-state film composition. This design feature facilitates seamless maintenance and replenishment of the solid-state film, ensuring continuous dispensing of the desired formulation. By integrating a replaceable cartridge or roll within the housing, the invention optimizes convenience and efficiency, allowing users to easily swap out depleted film components without disrupting the device's operation.

In an embodiment, the mechanism lies a pivotal element designed to respond to user input-a splitter or cutter specifically engineered to split or cut the solid-state film composition. This crucial component enables precise control over dosage administration, allowing users to tailor their experience according to their preferences and requirements. By incorporating a splitter or cutter into the mechanism, the invention ensures accurate and consistent dispensing of the solid-state film, promoting efficiency and minimizing waste. This intuitive design feature enhances user convenience and satisfaction, offering a seamless and customizable experience across various applications in oral health, wellness, skincare, and pharmaceuticals.

In an embodiment, the invention discloses a rotary gear system to provide precise control over the dispensing of the solid-state composition. This intricate system employs gears that interlock and rotate with precision, enabling the exact modulation of the flow of the solid-state composition through the dispenser device. By utilizing a rotary gear system, the invention ensures consistent and accurate dosing, enhancing user experience and product effectiveness. This innovative design feature not only promotes efficiency but also facilitates customization, allowing users to adjust the dosage according to their specific needs. Whether in applications related to oral health, wellness, skincare, or pharmaceuticals, the rotary gear system guarantees reliable performance and optimal delivery of the solid-state composition, embodying a fusion of precision engineering and user-centric design.

In an embodiment, integral to the functionality of the invention is a sophisticated sensor system designed to detect the presence of the solid-state composition within the housing and relay this information to the user interface for dosage adjustment. This sensor system employs advanced technology to accurately monitor the level of the solid-state composition, ensuring that users are informed about the remaining quantity and prompting adjustments as necessary. By providing real-time feedback, the sensor system enhances user convenience and efficiency, enabling seamless management of dosage requirements.

In an embodiment, the housing of the invention is constructed from a UV-resistant material, a strategic choice aimed at safeguarding the solid-state composition from degradation caused by exposure to light. This purposeful selection ensures that the integrity and efficacy of the composition remain intact, even when the device is subjected to varying environmental conditions. By employing UV-resistant materials, the invention effectively shields the solid-state composition from harmful UV rays, which can lead to chemical alterations and diminish its potency over time. This protective measure underscores the commitment to maintaining product quality and longevity, ultimately enhancing user satisfaction and confidence in the device's performance. Whether in applications related to oral health, wellness, skincare, or pharmaceuticals, the UV-resistant housing serves as a reliable barrier against light-induced degradation, preserving the integrity of the solid-state composition and upholding its therapeutic or functional benefits.

In an embodiment, the invention discloses a spring-loaded plunger which ensures precise dosing by regulating the force applied to dispense the composition, thereby allowing users to administer the desired amount with accuracy and consistency. The spring-loaded plunger mechanism not only enhances user convenience but also promotes efficiency, minimizing waste and maximizing the utilization of the solid-state composition. Whether in applications pertaining to oral health, wellness, skincare, or pharmaceuticals, this reliable and intuitive component plays a pivotal role in optimizing the functionality and effectiveness of the dispenser device, epitomizing a synergy of precision engineering and user-centric design.

In an embodiment, the invention discloses a crucial locking mechanism to prevent unintended dispensing of the solid-state composition. This feature serves as a safeguard against accidental release, ensuring that the composition remains securely contained within the device until intentionally accessed by the user. By integrating a locking mechanism, the invention prioritizes user safety and minimizes the risk of waste or spillage. This element enhances user confidence and peace of mind, particularly in settings where precise control over dosage administration is paramount. Whether in applications related to oral health, wellness, skincare, or pharmaceuticals, the inclusion of a locking mechanism underscores a commitment to user-centricity and product reliability, contributing to a seamless and stress-free user experience.

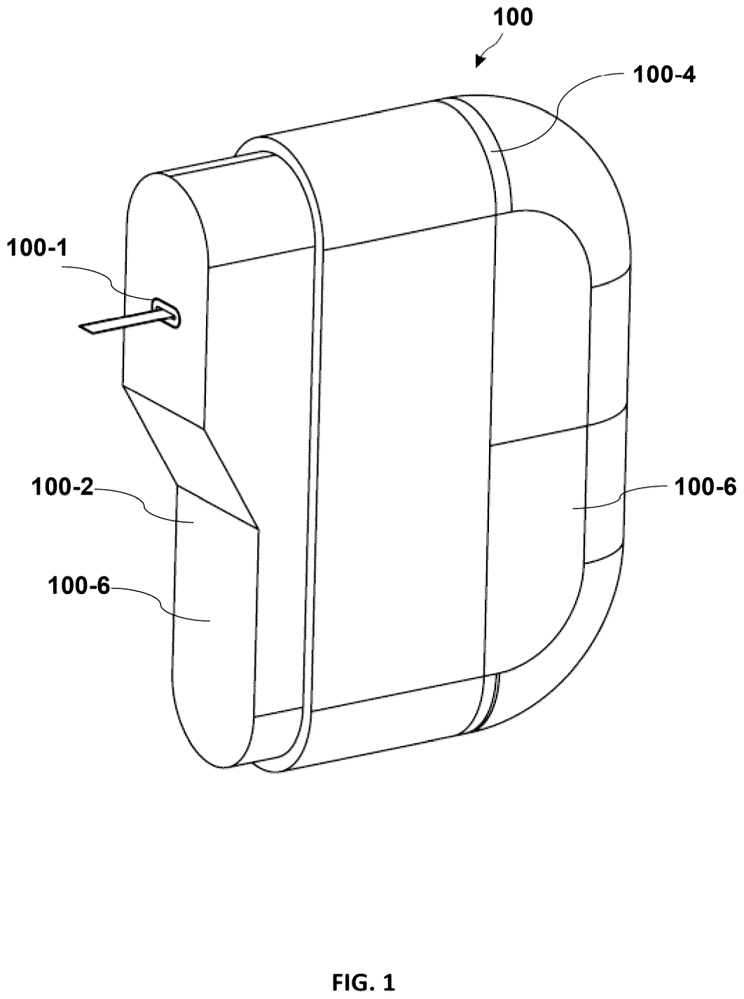

In accordance with an embodiment of the present invention, FIG. 1 is a schematic representation of the dispenser device 100 intended for administering a solid-state film composition is depicted. This illustration offers a visual overview of the device, highlighting its essential components and layout. It provides insight into the overall structure and design of the dispenser, including features such as the housing, mechanism for dispensing the solid-state film composition, and any accompanying elements pertinent to its functionality. The figure shows a solid-state film outlet 100-1 which is the exit point for the solid-state film composition, facilitating its controlled release. Further, a magnetic base 100-2 as shown in the figure provides stability and attachment for the device, ensuring secure positioning during operation. The dispenser trigger 100-3 is a user-operated mechanism that initiates the dispensing action, enabling users to release the film for administration. Further, there is shown a display 100-4 for the solid-state film offers visual feedback on the quantity or status of the film remaining in the dispenser, aiding users in monitoring usage. Additionally, a reserve solid-state film roll 100-5 serves as a backup supply, ensuring continuous availability of the film for administration.

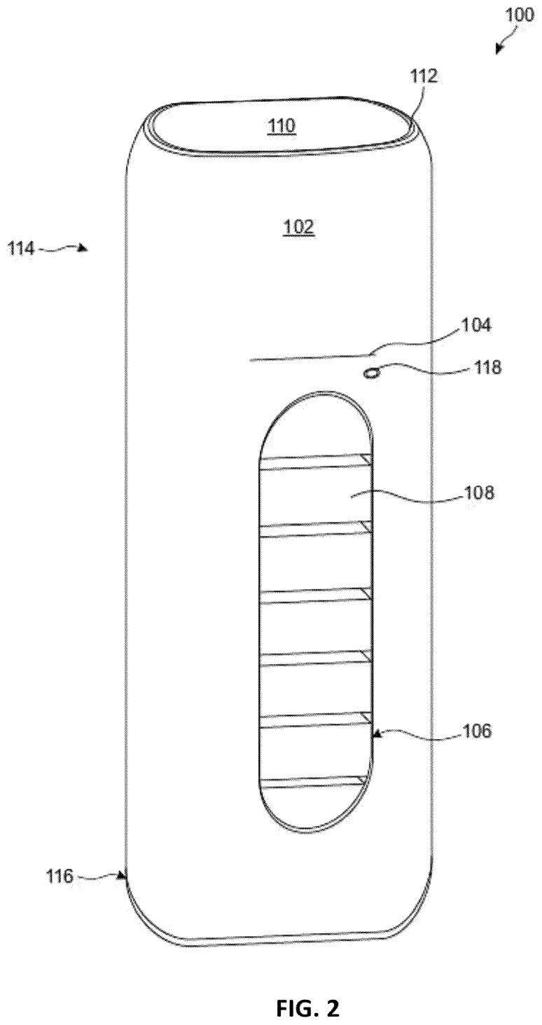

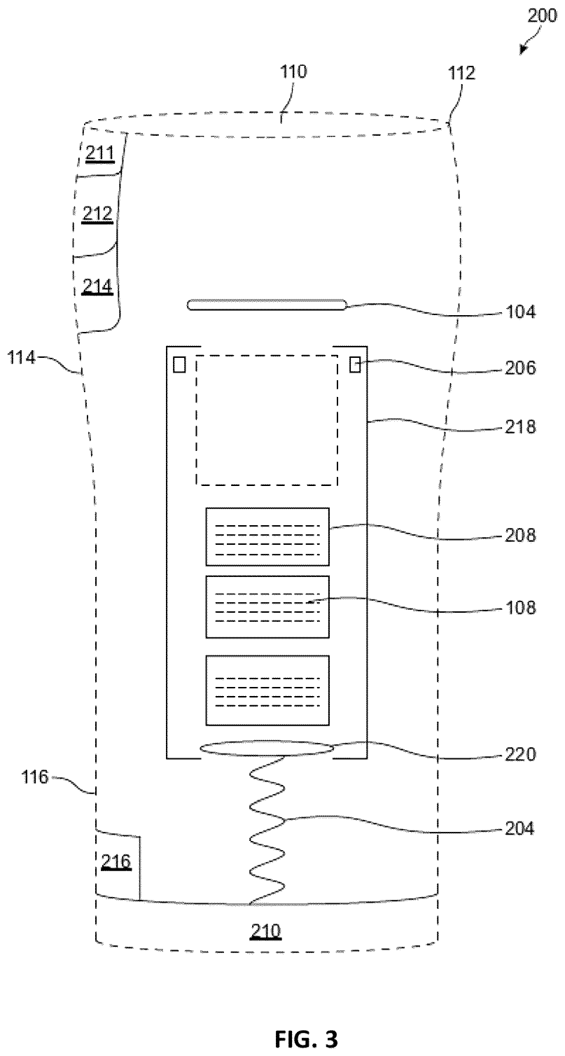

In accordance with an embodiment of the present invention, in FIG. 2, a front view of a device to dispense a solid-state film toothpaste, dietary supplements, skincare, and pharmaceuticals and the solid-state film is shown generally 100. The device 100 comprises housing 102, dispensing slot 104, housing interior 106 (shown in FIGS. 3-5), solid-state film 108, top or button 110, top section of housing 112, housing side section 114 and housing bottom section 116 and sensor 118.

The housing 102 has a hollow interior portion configured to house various components described herein, and other components that are ancillary to those described but not recited specifically (e.g., wiring, bearings, motor components, etc.). The housing 102 may be formed of various materials recycled materials and plastics. The housing 102 has a dispensing slot 104 configured to deliver, solid-state film toothpaste, dietary supplements, skincare, and pharmaceuticals, film 108 using the internal mechanisms described in relation to FIG. 3. As is shown in FIG. 2 the solid-state film toothpaste, dietary supplements, skincare, and pharmaceuticals film may be bunched, and or rolled together into bundles when compartmentalized, but may also just be stacked. The solid-state film toothpaste, dietary supplements, skincare, and pharmaceuticals film 108 are described in greater detail herein.

In some embodiments, a sensor 118 may be used to sense when a toothbrush is near the dispensing slot to automatically dispense a strip. In other embodiments, a mechanical finger tip button 110 that is depressible by a user to dispense a film 108. The device may be pocket size for travel but may also be a larger size for home use.

Referring now to FIG. 3, a vertical sectional view of a device to dispense a solid-state film toothpaste, dietary supplements, skincare, and pharmaceuticals in the device is shown at 200. For purposes of orientation, housing 102, dispensing slot 104, housing interior 106 and solid-state toothpaste 108, are shown. The housing 102 has a hollow interior 106 configured to house various components described herein, and other components that are ancillary to those described but not recited specifically (e.g., wiring, bearings, motor components, etc.).

Internal components of the device 100 can be seen herein including cartridge housing 218, cartridge aperture 202, motive clement such as a spring 204, cartridge holders 206, cartridges 208, and removable bottom 210. Circuitry and motors are also shown herein, including but not limited to processors 211, Bluetooth® or wireless communication 212, power inlet or batteries 214, and motor 216.

In some embodiments, cartridges of solid-state film toothpaste, dietary supplements, skincare, and pharmaceuticals 108 may be utilized and the device comprises a cartridge aperture 202 configured to accept the cartridge 208. While many mechanisms may be used to hold the cartridges 208 in place, holders 206 are shown and may comprise cars that hold the cartridges in place. However, other types of cartridge holders may be used to keep the cartridges 208 in place. In operation, the user can change the cartridges via a door or opening the housing on the outside, or via a removable top or removable bottom of the housing 102.

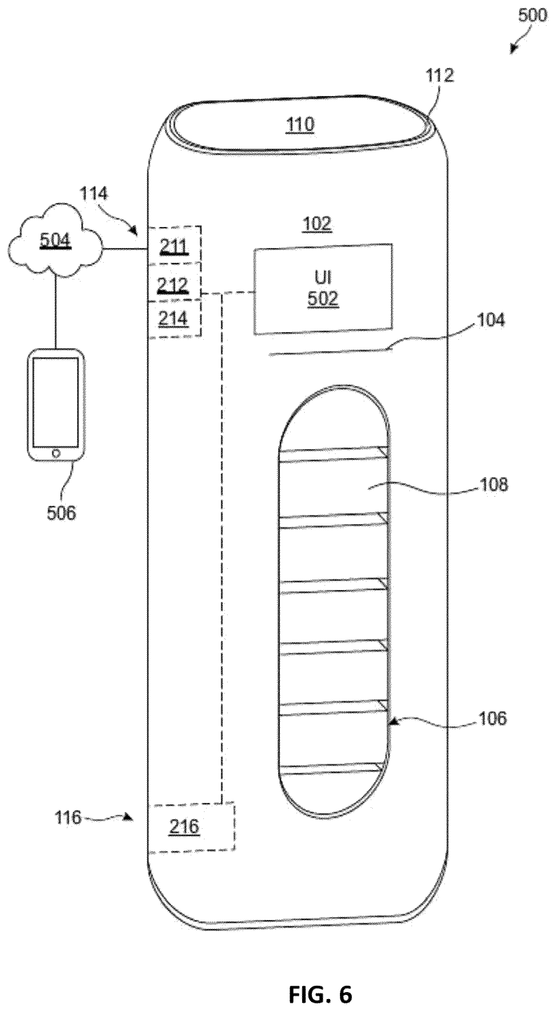

The motive clement 204 may be positioned toward the bottom section of the housing, and may comprise a spring, a plunger type mechanism, a telescoping arm, or any element that applies upward pressure to the cartridge housing 218 so that film 108 may be dispensed via dispensing slot 104. The motive clement 204 may be in communication or in contact with the motor 216, which is powered by batteries 216 and is actuated via sensor 118, or via a button when depressed by the user (discussed in greater detail with reference to FIG. 6). This will in turn cause the motive element 204 to apply pressure to the table 220 and trigger the cartridges outward to dispense the film 108.

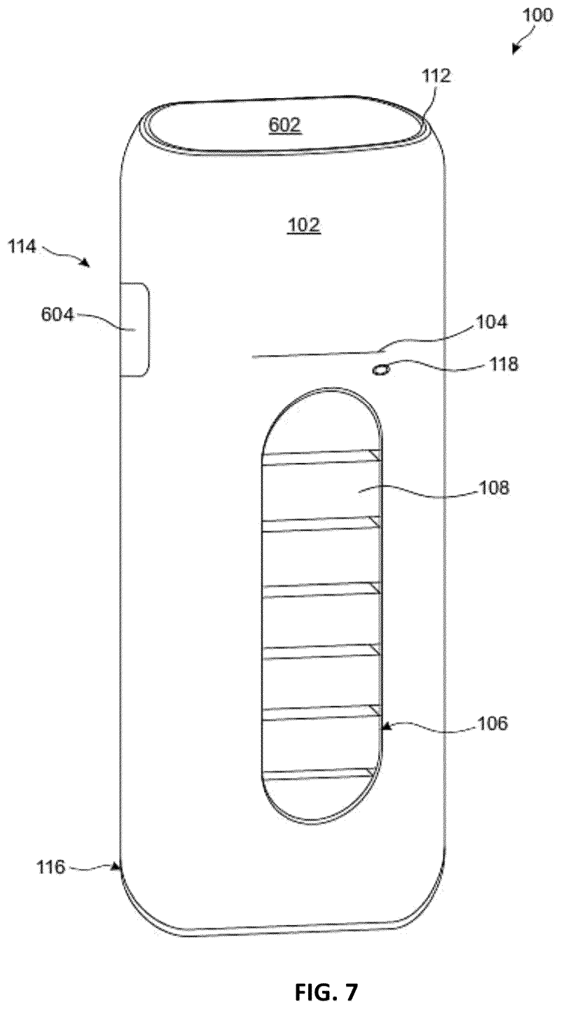

The processor 211, Bluetooth® or wireless communication 212, may be in communication with the motor 116 and sensor 118. In other embodiments, the device may be in contact with a user's smart phone via mobile application to control certain elements of the device 100. Furthermore, in embodiments discussed in relation to FIG. 7, a user interface may be employed on the device itself, so that a user can choose appropriate dosage and personalize the device.

In operation, in this particular embodiment, a digital input from a user's phone, a sensor or a button that actuates a motor may be employed. In other embodiments shown in relation to FIGS. 6-7, a purely mechanical trigger system may be used.

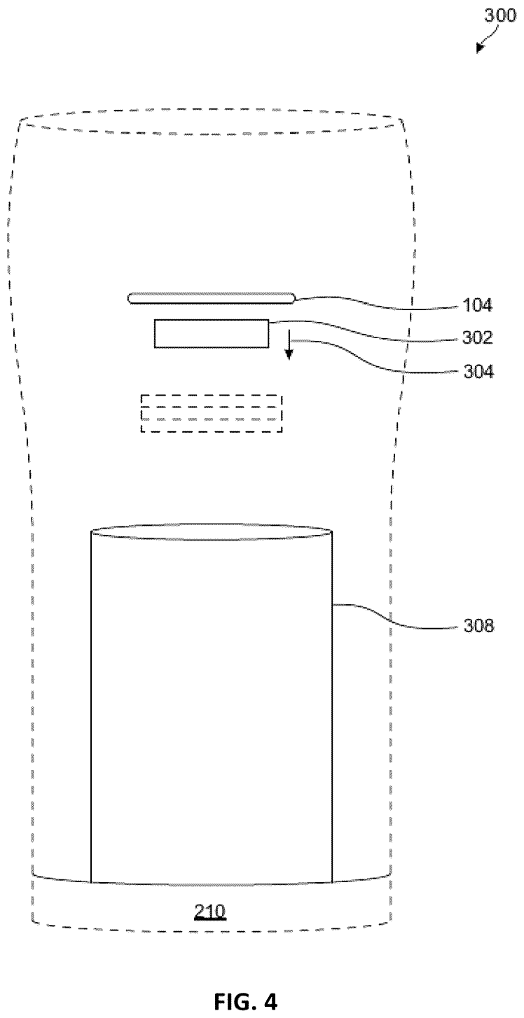

With reference now to FIG. 4 another vertical cross section view of a device to dispense a solid-state film toothpaste, dietary supplements, skincare, and pharmaceuticals and the film is shown generally at 300. In this embodiment, an exemplary cutting and dosing mechanism is shown, however, other mechanisms to ensure proper film dosage may be employed such as to predetermine the size of the strip, or separate housing for different sized film—or reserve film for children, and others.

For purposes of orientation, the dispensing slot 104 is shown and in interior is a cutting mechanism 302 that moves upward and downward as shown by arrow 304. The cutting mechanism 302 may be in communication with the processor and motor, such that a user can choose an appropriate dosage, a button on the outside, or a user interface on the outside of the housing of the device 100. Based on the dosage selected the cutting device can move in at least four directions to cut the film that is ready to be dispensed to a predetermined size based on the dosing requirement by the user. The rest of the film may either be used at a later time or fall into bin 308, which is configured to catch the portion of the film 118 that is not used.

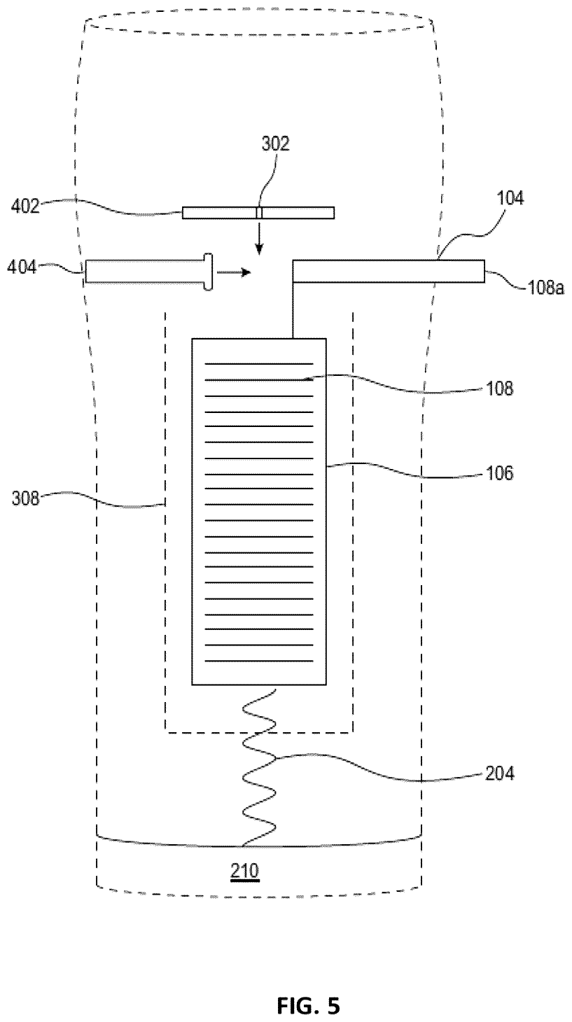

Referring now to FIG. 5, a side cross section view of a device to dispense a solid-state film toothpaste, dietary supplements, skincare, and pharmaceuticals and the solid-state film toothpaste, dietary supplements, skincare, and pharmaceuticals is shown generally at 400. For purposes of orientation, the cutting mechanism 302 is shown in bin 308, and motive element 204. This exemplary embodiment shows a side trigger in communication with either the processor or a mechanical button, shown with reference to FIG. 7. In this embodiment, the trigger is configured to push a toothpaste film 118a from the plurality of toothpaste film 108a out of the slot 104 for use by the user. Depending upon the dosage, the cutting mechanism 302 can slide in aperture 402 to cut the film into smaller film if the dosage is, for example, for a child. However, other types of mechanisms may be used to push or pull the film out of the housing 102.

With reference now to FIG. 6, a front view of a device to dispense a solid-state film toothpaste, dietary supplements, skincare, and pharmaceuticals and the solid-state film toothpaste, dietary supplements, skincare, and pharmaceuticals is shown generally at 500. The device 100 comprises housing 102, dispensing slot 104, housing interior 106 (shown in FIGS. 3-5), solid-state film 108, top or button 110, top section of housing 112, housing side section 114 and housing bottom section 116 and a user interface (UI) 502. The UI 502 may be in communication with the processor 211, Bluetooth® or wireless communication 212, power inlet or batteries 214, and motor 216. Users may be able to control the dosage and other elements of the device 100 including but not limited to Bluetooth® connectivity ordering additional strips, opening the door to replace the film and user notifications, for example. The device 100 may be connected to a network 504 in the user's smart phone or other smart device 506 to relay information back-and-forth from the device 100 to the smart phone 506 using a mobile application on the smart phone 506.

With reference now to FIG. 7, a side view of a device to dispense a solid-state film toothpaste, dietary supplements, skincare, and pharmaceuticals and the solid-state film toothpaste, dietary supplements, skincare, and pharmaceuticals is shown generally at 600. The device 100 comprises housing 102, dispensing slot 104, housing interior 106, solid-state film 108, top or button 110, top section of housing 112, housing side section 114 and housing bottom section 116. In this embodiment, the device is purely mechanical with no control circuitry, and relies on exemplary side buttons 604 or finger tip button 602. The mechanical aspects may include a variety of internal springs and plungers and gears configured to dispense the solid-state film toothpaste, dietary supplements, skincare, and pharmaceuticals. Other mechanical mechanisms may be utilized by the user to dispense the solid-state film toothpaste, dietary supplements, skincare, and pharmaceuticals.

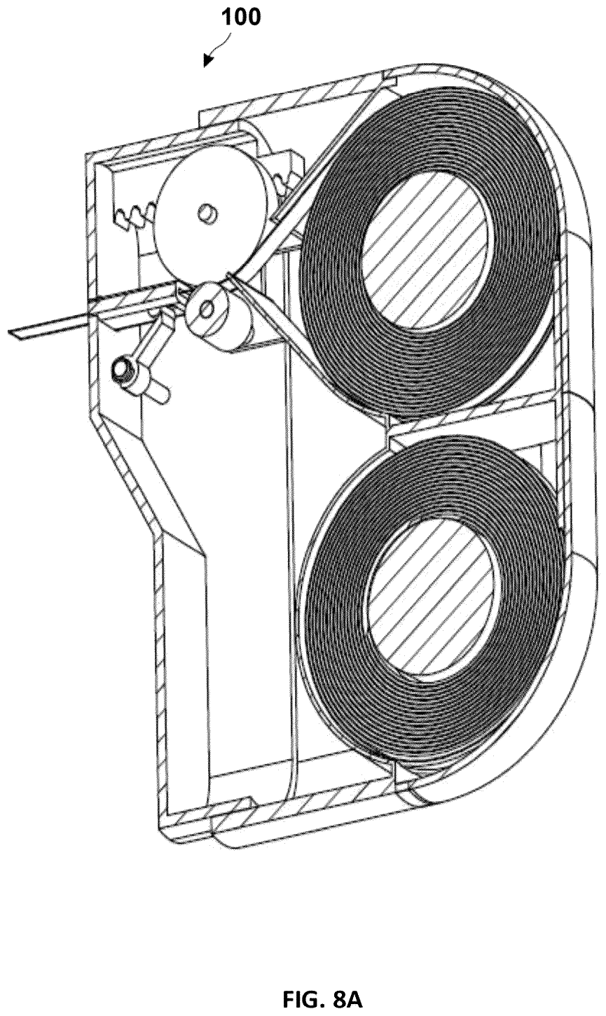

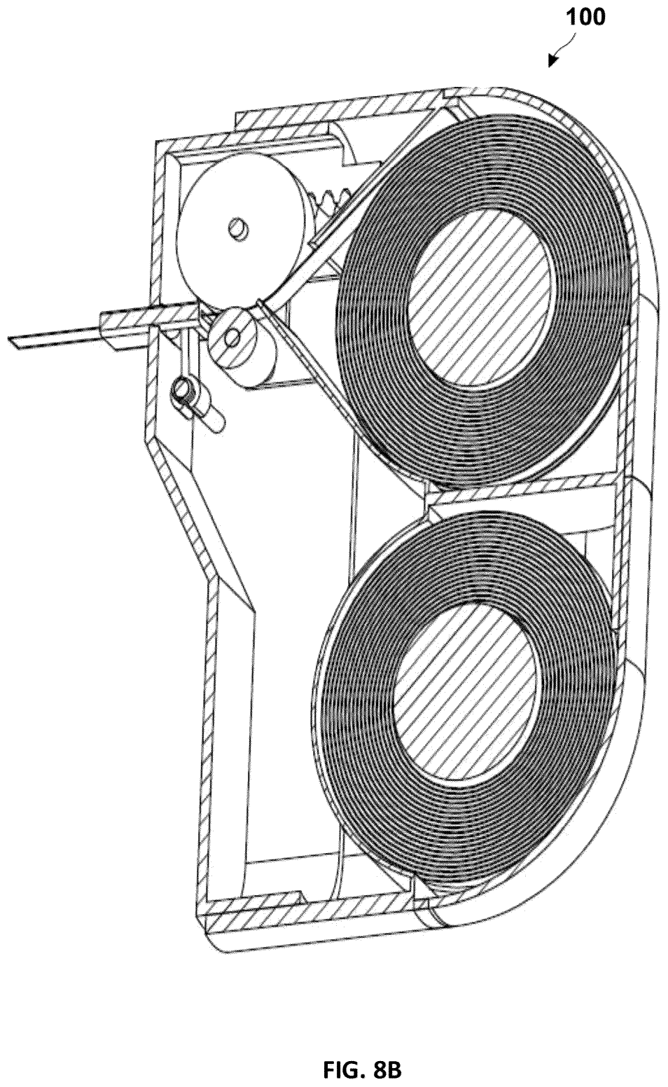

In accordance with an embodiment of the present invention, FIG. 8A and FIG. 8B is a cross-section view of the dispenser device. These illustrations provide an in-depth perspective on the internal structure and mechanisms of the device, revealing how its various components interact and contribute to its functionality. These figures show the internal layout of the device, including the arrangement of key elements such as the housing, dispensing mechanism, sensor systems, and any other pertinent features.

### Solid-State Film Composition

In one exemplary embodiment, which is not limiting in scope as many other ingredients may be used, the compositions for use in the device may be produced with glycerol, gelatin, flavoring, vitamins/minerals, glucose, purified water, sodium citrate, citric acid, breath freshening components, and various other dental hygiene, dietary supplementation, skincare, and pharmaceutical ingredients described herein.

The composition in its solid-state form may comprise a combination of pullulan, menthol, sucralose, glycerol oleate, copper gluconate, ceratonia siliqua, though other ingredients are contemplated.

The composition may comprise compounds and ingredients that have direct therapeutic benefits (e.g., active ingredients) and other components or ingredients that act as carriers or taste modifying ingredients (e.g., inactive ingredients).

The composition may comprise the active ingredients in the range from about 10.0% to about 40.0% by weight and inactive ingredients in the amount of 60% to about 90% by weight. In one embodiment, the composition comprises fluoropolymers such as for example, polytetrafluoroethylene (PTFE), fluorinated polyethylene-propylene (FEP), polyvinylidene fluoride (PVDF), and polyvinylfluoride (PVF). In embodiments, other anti-caries agents which enhances the dental remineralization process and helps repair early tooth decay, provide nutritional value, and prescription usage may be used.

In an embodiment, antimicrobial agents such as stannous fluoride, triclosan, and zinc citrate, which help to prevent the accumulation of plaque may be employed. Additionally, vitamins/minerals, pharmaceuticals may be used.

In an embodiment, flavorings such as peppermint, spearmint, cinnamon, wintergreen, and menthol may be used to freshen breath and add taste may be used. In other embodiments, culturally specific flavorings may be used.

In an embodiment, sweeteners such as xylitol, calcium or sodium saccharin and aspartame may be employed.

In an embodiment, the composition may be a microcrystalline cellulose and maltodextrin composition as a solid-state film and is quickly and completely digestible by the user.

Some of the non-limiting advantages of the present invention are:
Portability and Convenience: The dispenser device offers unparalleled convenience by eliminating the need for carrying traditional toothpaste and toothbrushes, making oral hygiene maintenance effortless, especially while traveling or on the go.
Precise Dosage Control: Users benefit from precise dosage control, facilitated by the adjustable mechanism, ensuring accurate dispensing of the solid-state film composition according to individual needs and preferences, thereby optimizing oral health outcomes.
Enhanced Hygiene: By utilizing a dispenser device, users can maintain optimal hygiene standards as the solid-state film compositions remain sealed within the device, protected from external contaminants and environmental factors, promoting overall oral health and wellness.
User-friendly Interface: Incorporation of a user interface enhances usability, allowing for intuitive selection of dosage and composition type, making the device accessible and easy to use for individuals of diverse backgrounds and capabilities.
Versatility in Composition: The dispenser device accommodates a variety of solid-state film compositions, including toothpaste formulations, dietary supplements, skincare products, and pharmaceutical formulations, providing users with a versatile solution for their oral care and wellness needs.

Terms and phrases used in this document, and variations thereof, unless otherwise expressly stated, should be construed as open-ended as opposed to limiting. As examples of the foregoing: the term “including” should be read as mean “including, without limitation” or the like; the term “example” is used to provide exemplary instances of the item in the discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. Although the foregoing subject matter has been described in some detail by way of illustration and example for purposes of clarity of understanding, it will be understood by those skilled in the art that certain changes and modifications can be practiced within the scope of the appended claims.

What is claimed is:

1. A dispenser device for administering a solid-state film composition, comprising:
a housing having an outlet;
a mechanism configured to advance the solid-state composition through an outlet of the housing, wherein the mechanism is adjustable to dispense precise dosages of the solid-state film composition, and
wherein the device comprises a user interface allowing users to select the dosage of solid-state film composition to be dispensed.

2. The dispenser device of claim 1, wherein the solid-state film composition comprises any one of a toothpaste formulation, or one or more dietary supplements configured for oral ingestion, or a skincare formulation for topical application, or a pharmaceutical formulation for medication delivery.

3. The dispenser device of claim 1, wherein the housing and mechanism are composed of materials non-reactive with the solid-state film composition.

4. The dispenser device of claim 1, wherein the housing comprises a replaceable cartridge or film roll containing the solid-state film composition.

5. The dispenser device of claim 1, wherein the mechanism comprises a splitter or a cutter to split or cut the solid-state film composition in response to user input.

6. The dispenser device of claim 1, wherein the mechanism comprises a rotary gear system for precise control of the solid-state composition dispensing.

7. The dispenser device of claim 1, further comprising a sensor system to detect the presence of the solid-state composition within the housing and provide feedback to the user interface for dosage adjustment.

8. The dispenser device of claim 1, wherein the housing is made of a UV-resistant material to protect the solid-state composition from degradation due to light exposure.

9. The dispenser device of claim 1, wherein the mechanism includes a spring-loaded plunger for controlled release of the solid-state composition.

10. The dispenser device of claim 1, further comprising a locking mechanism to prevent accidental dispensing of the solid-state composition.

11. The dispenser device of claim 1, wherein the user interface comprises a touchscreen display for intuitive control and monitoring of dosage and composition selection.

12. The dispenser device of claim 1, wherein the housing includes a compartment for storing additional cartridges of different solid-state compositions for easy interchangeability.

13. The dispenser device of claim 1, wherein the mechanism is equipped with temperature control features to maintain the stability of the solid-state composition.

14. The dispenser device of claim 1, further comprising a self-cleaning mechanism for the outlet to prevent clogging and ensure hygienic dispensing of the solid-state composition.

15. The dispenser device of claim 1, wherein the solid-state film outlet includes a valve mechanism for regulating the flow of the solid-state film composition.

16. The dispenser device of claim 1, wherein the magnetic base comprises an adjustable arm or extension for accommodating various surfaces and angles.

17. The dispenser device of claim 1, wherein the dispenser trigger includes a tactile feedback mechanism to provide users with confirmation of successful activation.
